(19) United States
(12) Patent Application Publication
(10) Pub. No.: US 2016/0136450 A1
(43) Pub. Date: May 19, 2016

(54) LASER SYSTEM AND METHOD FOR TREATING DIABETES

(71) Applicant: Therapeutic Laser Applications, LLC, Miami Lakes, FL (US)

(72) Inventor: Mark Chariff, Miami Lakes, FL (US)

(73) Assignee: THERAPEUTIC LASER APPLICATIONS, LLC, Miami Lakes, FL (US)

(21) Appl. No.: 14/925,814

(22) Filed: Oct. 28, 2015

Related U.S. Application Data
(60) Provisional application No. 62/081,536, filed on Nov. 18, 2014.

(51) Int. Cl. A61N 5/06; A61B 5/00; A61B 5/145

(52) U.S. Cl. CPC A61N 5/0613; A61B 5/14532; A61B 5/4836; A61N 2005/067

(57) ABSTRACT

A method of treating diabetes including activating a laser system, wherein the laser system emits a composite laser beam with more than one wavelength, and directing the composite laser beam over at least one of a pancreas, a thyroid, a foot, and a thoracic spine to treat diabetes.

### CROSS-REFERENCE TO RELATED APPLICATIONS

This application claims priority to U.S. provisional application 62/081,536, filed Nov. 18, 2014, which is herein incorporated by reference in its entirety.

### FIELD OF THE INVENTION

The disclosure generally relates to diabetic treatment using lasers.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Diabetes (i.e., diabetes mellitus) is a metabolic disease in which sufferers are unable to process excess blood sugar. The inability to process excess blood sugar may come from inadequate insulin production by the body, the improper response of the body's cells to insulin, or a combination of both. If left untreated, diabetes can cause serious bodily damage and even death. For example, unmanaged diabetes can increase the risk of cardiovascular disease as well as permanently damage blood vessels. Damaged blood vessels may lead to loss of eyesight, kidney failure, and nerve damage.

There are three main types of diabetes. Type 1 diabetes occurs when the pancreas fails to produce enough insulin to process excess sugar. Type 2 diabetes occurs when the body's cells fail to respond to insulin properly (i.e., the cells develop a kind of insulin resistance). The third type is gestational diabetes, which may develop in pregnant women. Depending on the type of diabetes and the seriousness of the condition, physicians may prescribe various treatment regimens. These treatment regimens typically include dietary changes, exercise programs, and medication (e.g., insulin injections, oral medication). Unfortunately, diabetes medications may be expensive and have undesirable side effects such as insulin resistance, weight gain, liver disease, etc.

### BRIEF SUMMARY OF THE INVENTION

The embodiments discussed below disclose a laser system and method that use laser light to stimulate cells in order to treat diabetes. Laser light consists of discrete wavelengths of coherent light from a narrow portion of the electromagnetic radiation spectrum (EMR). In general, it is amplified light that is monochromatic, collimated, and/or polarized that is then concentrated on a specific location (e.g., tissue). As will be explained below, some laser light is able to penetrate the skin surface with little or no heating of the biological tissue. As the laser light penetrates the skin surface, the laser light provides bio-stimulating energy to the body's cells enabling the cells to heal, regulate pain, grow, produce insulin, etc. Indeed, laser light therapy appears to restore biological systems to stable conditions (e.g., homeostasis).

It is believed that the energy from the laser system, in the form of photons, is absorbed by photo acceptor sites on a cell membrane. As the photonic energy is absorbed, the photonic energy triggers the cell's biochemical pathways, which initiate the transmission of a variety of signals that then start, inhibit, or accelerate a variety of biological processes. For example, laser light may enhance receptor-mediated movement across the cell membrane, which enables the cell to repair the cell's enzyme systems and re-establish the proper balance of proteins, ions, and/or carbohydrates within the cell. In some embodiments, laser light may increase the transport of ions, including calcium ions, across the cell membrane increasing the cell's ability to transmit signals. Laser light may also increase the levels of adenosine tri-phosphate (ATP) produced by the mitochondria of the cell. Specifically, the laser light may stimulate cytochromes (e.g., porphyrin) to produce singlet oxygen. Singlet oxygen in turn facilitates ATP creation. The increase in ATP enables more energy transportation within cells, which stimulate higher levels of cellular activity. In other words, increased ATP production increases cellular growth factors and higher levels of protein synthesis enabling cellular repair and functioning (e.g., produce insulin, etc.).

As will be discussed in detail below, the laser system produces a composite beam of laser energy emitted at wavelengths of approximately 532 nm (e.g., 500-594 nm), 808 nm (e.g., 780-980 nm), and 1064 nm (e.g., 1050-1300 nm) that uniquely stimulates the body's cells (e.g., pancreatic beta cells) to treat diabetes (e.g., produce insulin, absorb insulin). However, some embodiments may include a laser system that includes a combination of the three wavelengths (e.g., approximately 532 nm, 808 nm, and 1064 nm) that uniquely stimulate the body's cells to treat diabetes. In operation, it is believed that the composite beam penetrates deeply into biological tissue (e.g., 1, 2, 3, 4, 5, or more inches) without diverging; thus, enabling each component of the composite beam to simultaneously strike the same target tissue and at the same angle.

### BRIEF DESCRIPTION OF THE DRAWINGS

Various features, aspects, and advantages of the present invention will be better understood when the following detailed description is read with reference to the accompanying figures in which like characters represent like parts throughout the figures, wherein:

FIG. 1 is a cross-sectional view of an embodiment of a laser system;

FIG. 2 is a side view of a patient receiving diabetic treatment with a laser system; and

FIG. 3 is an embodiment of a method for treating diabetes with a laser system.

### DETAILED DESCRIPTION OF THE INVENTION

One or more specific embodiments of the present invention will be described below. These embodiments are only exemplary of the present invention. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

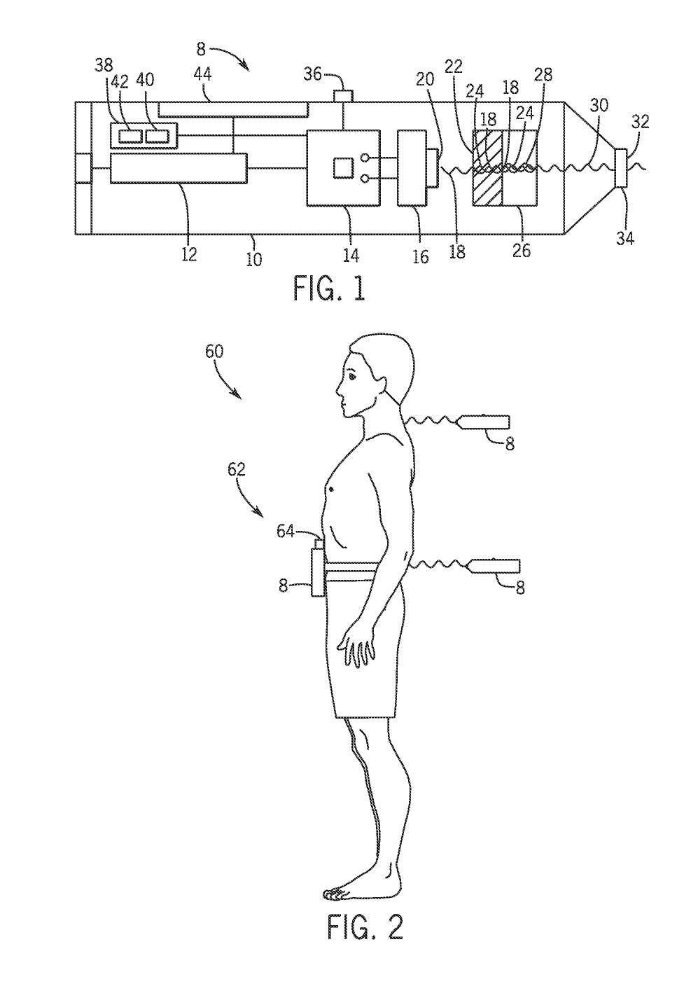

FIG. 1 is a cross-sectional view of an embodiment of a laser system 8. The laser system 8 may include a housing 10 that houses various components of the laser system 8. Components of the laser system 8 may include a power source 12 (e.g., batteries, standard plug-in electrical connection, or a combination thereof) that couples to a driver board 14 (e.g., 500 mW-5 W driver board). In operation, the power source 12 powers the driver board 14, which then powers a laser diode 16 (e.g., 808 nm laser diode). The diode 16, emits a first beam 18 (e.g., approximately 808 nm) through a microlens 20 and into a first crystal 22 (e.g., yttrium vanadate (NDYV04) crystal). As the first beam 18 passes through the first crystal 22, a portion of the first beam 18 changes into a second beam 24 (e.g., approximately 1064 nm) with a different wavelength. The second beam 24 and an unchanged portion of the first beam 18 then pass through a second crystal 26 (e.g., potassium titanium oxide (KTP) crystal) to form a third beam 28 (e.g., approximately 532 nm). The second crystal 26 then emits an unchanged portion of the first beam 18, an unchanged portion of the second beam 24, and the third beam 28 as a composite laser beam 30 (e.g., resultant composite laser beam) with the three different wavelengths (e.g., approximately 532 nm, 808 nm, and 1064 nm). After exiting the second crystal 26, the composite laser beam 30 passes through a collimating lens 32, and then out of the laser system 8 through an aperture 34 (e.g., single aperture). In some embodiments, the laser system 8 may not include a focusing lens in order to produce a more diffuse beam. A more diffuse beam may minimize the thermal effects from higher wavelength portions of the composite laser beam 30.

As illustrated, the laser system 8 may include an on/off switch 36 that enables an operator to control power transmission. Thus, enabling a user to regulate the duration of the treatment. In some embodiments, the laser system 8 may include a controller 38 that controls operation of the laser system 8. The controller 38 may include a processor 40 specifically programmed to execute instructions stored (e.g., programs) on a memory 42 to control the power source 12, driver board 14, and/or display 44. For example, the controller 38 may pulse the composite beam (e.g., 1 Hz to 5 kHz), change power (e.g., 500 mW to 5 W), implement a specific treatment protocol, etc. In some embodiments, the laser system 8 may provide feedback through a display, meters, and/or gauges 44 that enable a user to understand an operating condition of the laser system 8. For example, the display, meters, and/or gauges 44 may display a status of the laser system 8 including the wavelengths of emitted light, power level, treatment regimen, timer, pulse frequency, etc.

While the laser system 8 includes a single laser diode 16, some embodiments may include multiple diodes 16 (e.g., 1, 2, 3, 4, 5, or more). For example, the laser system 8 may include a diode for each desired wavelength (e.g., approximately 532 nm, 808 nm, and 1064 nm) and a beam combiner that then combines the beams from the different diodes into a composite laser beam. In operation, the laser system 8 may enable selective activation and power output from each of the diodes 16. For example, some treatment regimens may call for more laser light from a specific wavelength (e.g., approximately 532 nm, 808 nm, or 1064 nm). In these situations, a user or the controller 38 may increase the power of a particular diode 16 and/or use the remaining diodes 16 for less time during a treatment.

FIG. 2 is a side view of a patient receiving diabetic treatment with the laser system 8. As explained above, the laser system 8 treats diabetes. In some embodiments, the laser system 8 may be directed/focused over a specific portion of the body (e.g., pancreas, thyroid, thoracic spine, feet, etc.) to treat diabetes. For example, by focusing the composite beam 30 onto or in vicinity of the pancreas, the laser system 8 may stimulate increased production of insulin by the body. Likewise, by focusing the composite beam 30 onto the thyroid, the laser system 8 may stimulate the thyroid's ability to manage energy, protein production, etc.

In some embodiments, the laser system 8 may be a wearable system worn by a patient 60 around a specific portion of the body (e.g., like an insulin pump). For example, the laser system 8 may be worn by the patient 60 in vicinity of the pancreas using a strap or other wearable device. In some embodiments, the patient may wear a diabetes treatment system 62 that includes the laser system 8 coupled (e.g., wired, wirelessly) to one or more diabetes sensors or monitors 64 (e.g., epidermal glucose sensor, subcutaneous glucose sensor). In operation, the controller 38 may receive input from the diabetes sensor 64 regarding the glucose levels of the patient 60. If the glucose levels of the patient 60 are high, the controller 38 activates the laser system 8 to produce the composite laser beam 30. The composite laser beam 30 then stimulates insulin production by the pancreas, which reduces blood sugar levels.

As explained above, the controller 38 may include stored laser treatment regimens. These regimens may be based on specific glucose levels, patient size (e.g., amount of fat tissue between the laser system 8 and a target tissue), time since last treatment, etc. For example, the detection of high glucose levels may trigger an extensive laser treatment (e.g., longer duration, more intense laser beam, or a combination thereof) by the laser system 8. Similarly, moderate levels of glucose may trigger a less extensive laser treatment (e.g., shorter duration, less intense laser beam, etc.) by the laser system 8. In some embodiments, the regimen may include waiting a predetermined time period before starting the laser treatment again and/or the laser system 8 may wait for another glucose measurement before restarting the laser treatment.

As explained above, laser treatment by the laser system 8 may vary in duration, power, intensity, location, and distance from the patient 60. For example, one treatment regimen may involve irradiating the pancreas, thyroid, foot, thoracic spine, etc. in a scanning pattern approximately one inch away from a patient's skin with a composite laser beam 30 at approximately three hundred sixty mW and pulsed at approximately thirty Hz for approximately two minutes. The regimen may then irradiate the pancreas, thyroid, foot, thoracic spine, etc. with a continuous composite laser beam 30 for approximately one minute at approximately three hundred sixty mW. However, this is only an exemplary treatment regimen and other regimens may increase or decrease the power, frequency of pulses, time, distance, laser movement pattern, etc.

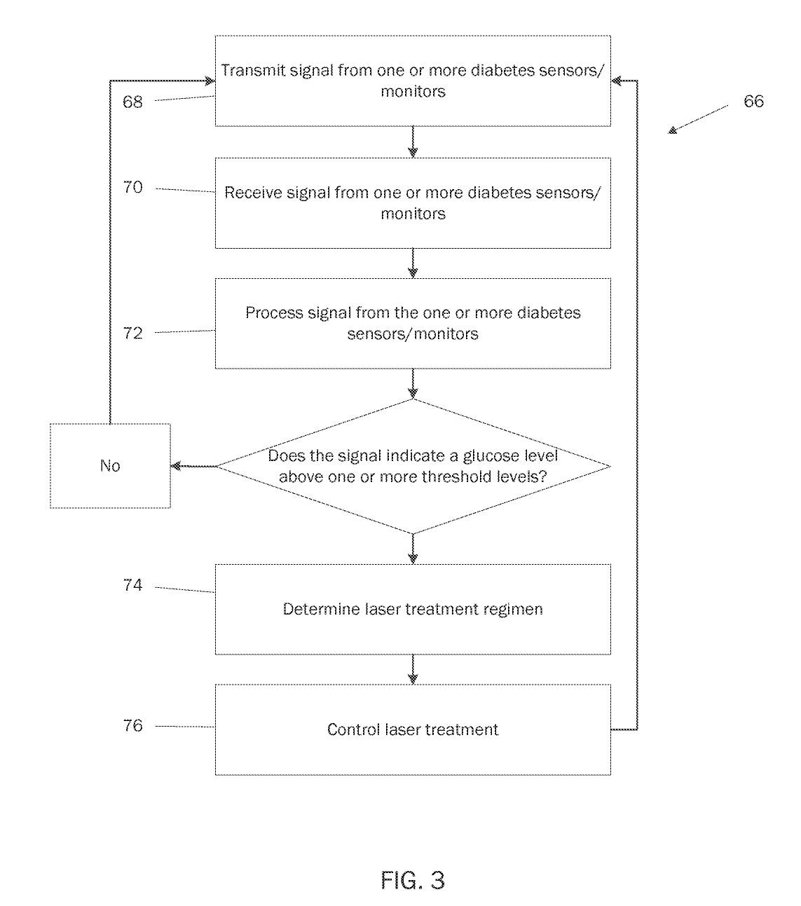

FIG. 3 is an embodiment of a method 66 for treating diabetes with a laser system 8. The method begins by transmitting a signal from one or more diabetes sensors and/or monitors 64 (step 68). In some embodiments, the diabetes sensors and/or monitors 64 may transmit signals periodically, when requested by a user, and/or a combination thereof. For example, the diabetes sensors or monitors 64 may transmit a signal every hour, every six hours, before scheduled meal times, after scheduled meal times, etc. A controller (e.g., controller 38) receives the signal from the one or more sensors or monitors 64 (step 70) and then processes the signal using a processor (e.g., 40) (step 72). The processor may be specifically programmed to execute instructions stored on a memory (e.g., 42) to determine what kind of glucose level the signal represents as well as other steps in the method 66.

If the signal represents a normal glucose level, the method returns to transmitting and receiving signals from one or more diabetes sensors 64 (steps 68 and 70). If the signal represents a glucose level above one or more threshold levels (e.g., 1, 2, 3, 4, 5, or more), the processor determines the type of laser treatment regimen that will bring the glucose levels to a normal and/or acceptable level (step 74). For example, if the processor determines that the glucose levels are above a first threshold level but below a second threshold level, the processor may determine that a first treatment regimen among one or more treatment regimens (e.g., 1, 2, 3, 4, 5 treatment regimens) will bring the glucose level to a normal and/or acceptable level. In some embodiments, if the glucose level is above the second threshold level but below a third threshold level, the processor may determine that a second treatment regimen should be used. Similarly, some embodiments may include additional treatment regimens (e.g., 1, 2, 3, 4, 5, or more treatment regimens) that treat one or more glucose threshold levels (e.g., 1, 2, 3, 4, 5, or more thresholds). As explained above, treatment regimens may differ in time, motion of laser, laser intensity, frequency of laser pulses, laser wavelengths, and/or amount of time the target is exposed to different wavelengths. Once the type of laser treatment has been determined the controller controls the laser treatment system 8 with the processor (step 76). After finishing the treatment, the method 66 returns to transmitting and receiving signals from one or more diabetes sensors (steps 68, 70) to determine whether the user would benefit from additional laser treatments.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

What is claimed is:

1. A diabetes treatment system, comprising:
a glucose sensor configured to detect a glucose level in a patient; and
a laser system coupled to the glucose sensor, wherein the laser system is
configured to treat diabetes by emitting a composite laser beam with more than one wavelength in response to the detected glucose level.

2. The system of claim 1, wherein the composite laser beam comprises a first laser beam with a first wavelength of approximately 532-594 nm.

3. The system of claim 1, wherein the composite laser beam comprises a second laser beam with a second wavelength of approximately 808-980 nm.

4. The system of claim 1, wherein the composite laser beam comprises a third laser beam with a third wavelength of approximately 1064-1300 nm.

5. The system of claim 1, comprising a controller configured to control the laser system in response to a signal from the glucose sensor.

6. The system of claim 5, wherein the controller comprises a processor, and a memory configured to store one or more treatment regimens, and wherein the controller selects a treatment regimen based on the signal from the glucose sensor.

7. The system of claim 1, wherein the laser system comprises one or more laser diodes.

8. The system of claim 1, wherein the diabetes treatment system is configured to couple to a user.

9. A method of treating diabetes, comprising:
activating a laser system, wherein the laser system emits a composite laser beam with more than one wavelength; and
directing the composite laser beam over at least one of a pancreas, a thyroid, a foot, and a thoracic spine to treat diabetes.

10. The method of claim 9, wherein the composite laser beam comprises a first laser beam with a first wavelength of approximately 532-594 nm.

11. The method of claim 9, wherein the composite laser beam comprises a second laser beam with a second wavelength of approximately 808-980 nm.

12. The method of claim 9, wherein the composite user beam comprises a third laser beam with a third wavelength of approximately 1064-1300 nm.

13. The method of claim 9, comprising pulsing the composite laser beam.

14. The method of claim 9, comprising moving the composite laser beam in a pattern over at least one of the pancreas, the thyroid, the foot, and the thoracic spine.

15. The method of claim 9, comprising directing the composite laser beam for a first duration over at least one of the pancreas, the thyroid, the foot, and the thoracic spine.

16. A method of treating diabetes, comprising:
receiving a signal from a glucose sensor; and
controlling a laser system in response to the signal from the glucose sensor, wherein the laser system emits a composite laser beam with a first laser beam, a second laser beam, and a third laser beam, wherein the first laser beam has a first wavelength, the second laser beam has a second wavelength, and the third laser beam has a third wave length, and wherein the first wavelength, the second wavelength, and the third wavelength are different from one another.

17. The method of claim 16, comprising directing the composite laser beam over at least one of a pancreas, a thyroid, a foot, and a thoracic spine to treat diabetes.

18. The method of claim 16, wherein the first wavelength is approximately 532-594 nm.

19. The method of claim 16, wherein the second wavelength is approximately 808-980 nm.

20. The method of claim 16, wherein the third wavelength is approximately 1064-1300 nm.
